(19) United States
(12) Patent Application Publication
(10) Pub. No.: US 2025/0375147 A1
(43) Pub. Date: Dec. 11, 2025

(54) SEIZURE DETECTION

(71) Applicant: Samsung Electronics Co., Ltd., Suwon-si (KR)

(72) Inventors: Quan Dong, Pasadena, CA (US); Chaoyi Kang, Porter Ranch, CA (US); Yu Hao Chang, Burlingame, CA (US); Matthew Wiggins, San Jose, CA (US)

(21) Appl. No.: 18/812,491

(22) Filed: Aug. 22, 2024

Related U.S. Application Data
(60) Provisional application No. 63/658,742, filed on Jun. 11, 2024.

(51) Int. Cl. A61B 5/00; A61B 5/0205; A61B 5/024; A61B 5/03; A61B 5/11; A61B 5/1455; A61B 5/296

(52) U.S. Cl. CPC A61B 5/4094; A61B 5/0205; A61B 5/7221; A61B 5/7267; A61B 5/02416; A61B 5/031; A61B 5/11; A61B 5/14551; A61B 5/296; A61B 2560/0462; A61B 2562/0219

(57) ABSTRACT

In one embodiment, a method includes detecting, by each of multiple sensors of a wearable device worn by a user, a physiological signal of the user; determining, by the wearable device, whether at least one of the detected physiological signals indicates that the user is suffering a seizure; and in response to a determination that the user is suffering a seizure, then determining, by a trained neural network and based on multiple detected physiological signals, a seizure condition of the user.

### PRIORITY CLAIM

This application claims the benefit under 35 U.S.C. §119 of U.S. Provisional Patent Application No. 63/658,742 filed Jun. 11, 2024, which is incorporated by reference herein.

### TECHNICAL FIELD

This application generally relates to detecting seizures.

### BACKGROUND

Seizures occur due to abnormal neuronal activity in a person's brain. Symptoms of seizures include uncontrolled, involuntary shaking, confusion, and loss of consciousness, and different seizures have different symptoms. Seizures may last from a few seconds to several minutes.

Epilepsy is a central nervous system disorder in which brain activity becomes abnormal, causing seizures or periods of unusual behavior, sensations and sometimes loss of awareness. Epilepsy is one of the most common neurological diseases globally. The epilepsy patient population has a high rate of premature death due to Sudden Unexpected Death in Epilepsy (SUDEP). Risk factors include uncontrolled or frequent seizures, generalized convulsive seizures, and missing doses of medicine.

### BRIEF DESCRIPTION OF THE DRAWINGS

FIG. 1 illustrates an example method that uses a wearable device to detect a seizure or a seizure-related condition of a user.

FIG. 2 illustrates an example architecture of a system that includes the wearable device.

FIG. 3 illustrates an example computing system.

### DESCRIPTION OF EXAMPLE EMBODIMENTS

Seizures can range from mild to life threatening, can occur at any time of day or night, and can vary widely in frequency (e.g., from less than once a week to many times a day). Seizure monitoring is typically performed by visually observing a person, or through a combination of equipment and health-care professionals in a clinical setting. However, these approaches are human intensive and impractical for the majority of a person's life. In addition, certain dangerous conditions such as SUDEP occur at night, when monitoring is difficult and may be uncomfortable and disruptive to the person's sleep.

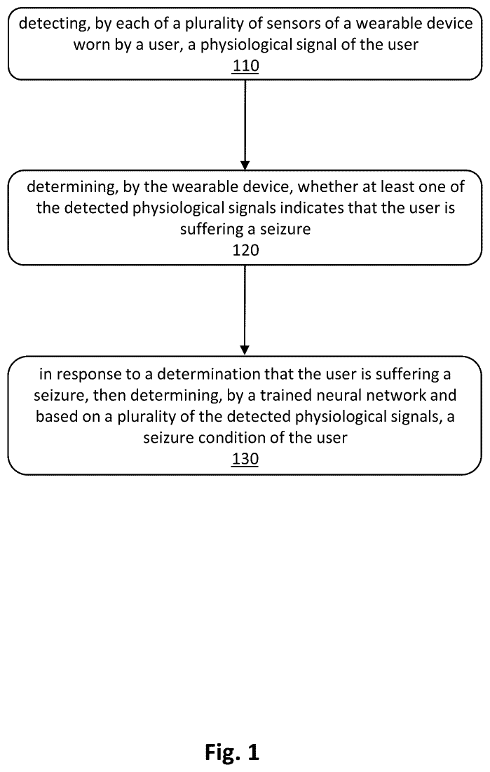

FIG. 1 illustrates an example method that uses a wearable device to detect a seizure or a seizure-related condition of a user. Step 110 of the example method of FIG. 1 includes detecting, by each of multiple sensors of a wearable device worn by a user, a physiological signal of the user. The wearable device may be a watch, or may be another wrist-worn device such as a bracelet. In particular embodiments, the wearable device may be a ring, a necklace or pendant, a chest-worn device, a head-worn device (such as earbuds, glasses, or a head-mounted device), or any other suitable wearable device.

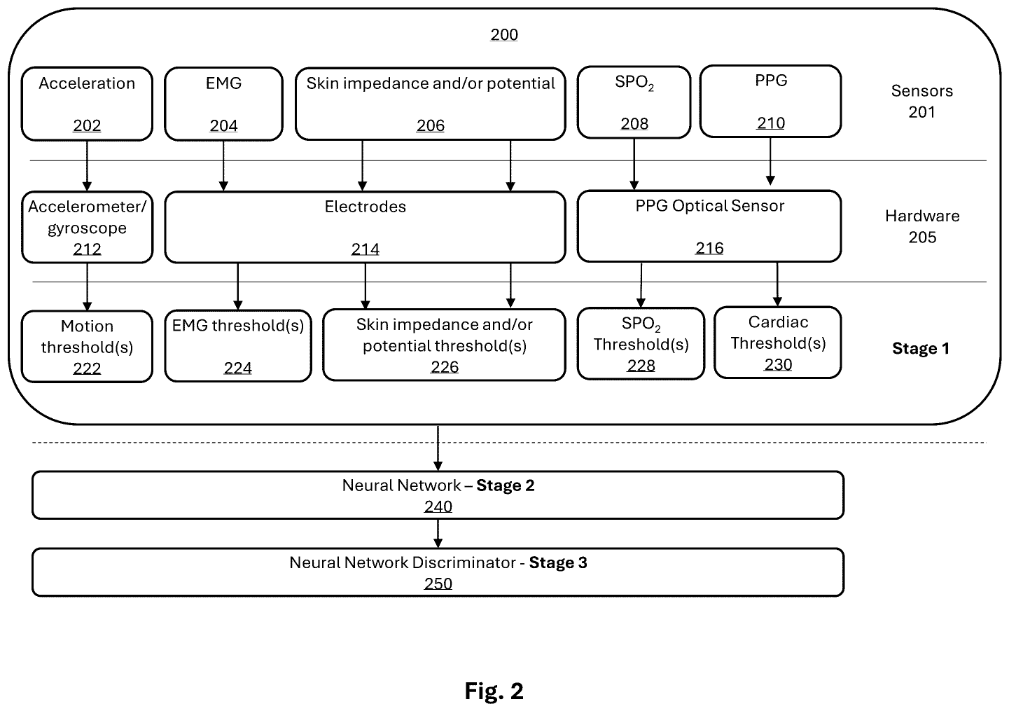

The wearable device includes multiple sensors that can detect a physiological signal of the user wearing the wearable device. FIG. 2 illustrates an example architecture of a system that includes a wearable device 200. As illustrated in FIG. 2, a wearable device includes example sensors 201, which are in-part realized by specific hardware 205. For example, wearable device 200 includes an acceleration sensor 202, which may be realized by one or more of an accelerometer or gyroscope 212. Wearable device 200 includes an EMG sensor 204, which may be realized by electrodes 214 on wearable device 200 (e.g., on a portion of the wearable device contacting the user's skin, such as on the back of a watch). Wearable device 200 includes a skin impedance and/or skin potential sensor 206, which may be realized by electrodes 214. While a single set of electrodes 214 are shown as providing hardware for EMG sensors 214 and skin impedance/potential sensors 206, different electrodes may be used for those sensors. Wearable device 200 includes SpO₂ sensor 208 and a PPG sensor 210, each of which may be realized by a PPG optical sensor 216.

Different sensors detect different physiological signals, and these signals vary based on the physiological state of the user. For example, acceleration sensor 212 can detect motion and body orientation (e.g., posture), EMG sensor 204 can detect muscle contractions, skin impedance/potential sensor 206 can detect sympathetic nerve activity and electrodermal activity, SpO₂ sensor 208 can detect blood oxygen, and PPG sensor 210 can detect cardiac-related signals, such as heart rate, tachycardia, asystole, and sudden RR interval variability change.

Seizures manifest themselves through various physiological signals, but merely using a sensor to track a physiological signal (e.g., using an accelerometer to detect shaking) results in poor sensitivity and poor specificity for seizure detection. For example, sudden changes along an axis of an accelerometer can indicate a fall, and spikes in motion at particular frequencies can indicate shaking, each of which can be symptoms of certain kinds of seizures. But sudden changes in accelerometer data can be caused by a number of different non-seizure activities (e.g., exercising, playing sports, shaking an object, etc.), and both falling and shaking occur apart from seizures. Therefore, merely tracking each of multiple physiological signals associated with seizures will result in many false positives or many false negatives, and likely both.

The techniques described herein overcome these problems and achieve both high specificity and high sensitivity using a wearable device through a two-stage approach to seizure detection. In the first stage, step 120 of the example method includes determining, by the wearable device, whether at least one of the detected physiological signals indicates that the user is suffering a seizure. As explained herein, this stage has high sensitivity, in that stage 1 is met when any one physiological signal indicates that the user is having a seizure. This results in poor specificity, but as explained below, the overall two-stage approach corrects for this, resulting in a seizure-detection approach that has both high specificity and high sensitivity. Moreover, as explained below, this approach is efficient in both power consumption and compute resources, as stage 1 is relatively low power relative to stage 2, and requires much less processing resources than does stage 2.

The wearable device determines whether at least one of the detected physiological signals indicates that the user is suffering a seizure by comparing each physiological signal detected by a sensor to a corresponding threshold for that particular physiological signal. As explained below, the thresholds are specific to the physiological signal, in that each physiological signal has a different associated threshold. For instance, as illustrated in FIG. 2, each physiological signal is associated with a specific corresponding threshold, e.g., motion threshold 222, EMG threshold 224, skin impedance/potential threshold 226, SpO₂ threshold 228, and cardiac threshold 230. Thresholds may include particular signatures, e.g., a recorded signal may be compared to a stored signal that indicates a seizure, and the threshold may require that the recorded signal be less than a maximum allowable deviation from the recorded signal.

Thresholds may focus on particular aspects of a sensor signal, such as the min, max, standard deviation, spectrum, noise, etc. of a signal. For example, a stage-1 threshold for an accelerometer sensor may be a magnitude of a sudden change in an accelerometer signal along an axis of the accelerometer's frame of reference, indicating that the user has a fallen. Another stage-1 threshold may be the frequency at which a particular motion occurs (e.g., as determined by a number of crossings of an accelerometer signal across an axis). As another example, for a galvanic skin response sensor, a stage-1 threshold may be a particular minimum rise in potential over a certain period of time. As another example, a stage-1 threshold for an EMG sensor may include a correlation coefficient between the EMG measurement and predetermined seizure muscle contraction templates that exceeds a minimum correlation coefficient threshold. As another example, a stage-1 threshold for an SpO₂ sensor may include an oxygen saturation dip that exceeds a threshold amount or a maximum duration of low oxygen saturation. As another example, a stage-1 threshold for a PPG sensor may include a heart rate change or beat-to-beat variance that exceeds a corresponding threshold change or variance, a long pause, or too high/low a heart rate. Particular embodiments may use multiple stage-1 thresholds for a single sensor (e.g., because a type of seizure can manifest multiple symptoms, and because different seizures may manifest different symptoms), such that if any one threshold is met, then the system proceeds to stage 2. In particular embodiments, multiple stage-1 thresholds may need to be met in order for the system to proceed to stage 2, although this may reduce sensitivity in stage 1.

In particular embodiment, a stage-1 threshold for a particular sensor may be tuned to the user of the wearable device, for example by defining a threshold value based on the user's particular characteristics. For example, the user's age, comorbidities, and/or known seizure types associated with the user may be used to define a value of a threshold for a particular sensor in stage 1. For example, because stage 1 is more associated with sensitivity, if a user is known to suffer physical shaking in their seizures, then the stage-1 threshold for motion at particular shaking frequencies may be relatively lower for that user.

In particular embodiments, in order for the wearable device to determine whether a particular physiological signal indicates that the user is suffering a seizure, the wearable device first ensures that the detected physiological signal is of sufficiently high quality. For example, the wearable device may apply a quality check for each physiological signal to determine whether that signal can be used for stage 1 detection. If a particular physiological signal meets its quality check, then it can be used during stage 1. If a particular physiological signal does not meet its quality check, then the signal is discarded from the stage 1 analysis for the time being. Since the wearable device is periodically collecting data (e.g., effectively continuously, or otherwise relatively frequently), a physiological signal that does not meet its quality check for stage 1 at a particular time may meet it at a different, even nearby time, and vice versa.

In particular embodiments, the stage-1 signal-quality checks are relatively lightweight, in keeping with stage 1's low-power, high sensitivity attributes. For example, stage-1 signal-quality checks include but are not limited to checking for motion artifacts, signal clipping, abrupt signal changes beyond physiological changes, and excess noise outside the signal spectrum.

Whenever any sensor signal in stage 1 meets its stage-2 triggering conditions (e.g., meets the threshold value for that sensor signal), then stage 2 occurs. For example, step 130 of the example method of FIG. 1 includes in response to a determination that the user is suffering a seizure, then determining, by a trained neural network and based on multiple detected physiological signals, a seizure condition of the user. In stage 2 and in the example of FIG. 2, data from each of the sensor signals is sent to a neural network 240. In stage 1, data is buffered for a period of time (e.g., the past 30 minutes of data from each sensor is buffered), so that if stage 2 is triggered, then the buffered data (along with data recorded after stage 2 is activated) can be sent to the neural network. The neural network may be deployed on the wearable device, or may be deployed on a different device (e.g., a client device such as a smartphone or a server device). The neural network in stage 2 can include multiple neural networks, as explained below.

In particular embodiments, a stage-2 quality check may be implemented for each sensor signal before that signal is used in stage 2 (i.e., before the signal is input to the neural network). If a particular signal does not meets its stage-2 quality check, then that signal is not used for the stage-2 analysis. In particular embodiments, a minimum number or percentage of sensor signals may need to meet their respective stage 2 quality checks in order for stage 2 to be performed.

Stage 2 quality checks may be more comprehensive than the stage 1 quality checks, to ensure that the measurement accurately reflects physiological conditions. For example, stage 2 can check the possibility of missing beats through PPG measurement due to low perfusion. Once the checker detects frequent missing beats, the heart rate measurement is flagged as inaccurate. As an alternative input, the stage 2 algorithm takes the signal quality check output that the perfusion is so low that heartbeats cannot be detected reliably.

In stage 2, features are extracted from each sensor signal, and these features are input to the neural network. The features from each sensor signal may be concatenated into a single feature vector having multiple cells identifying the various features. Then, the neural network outputs a predicted seizure condition of the user, based on the neural network's training. In stage 2, unlike in stage 1, this prediction is based on multiple input sensor signals (i.e., on each sensor signal that passes its stage-2 quality check), and therefore specificity is relatively high.

In particular embodiments, the neural network outputs a binary classification (e.g., “yes/no”) as to whether the user experienced or is experiencing a seizure. In particular embodiments, the neural network outputs a classification that identifies a particular type of seizure, which may be accompanied by a probability that the user experienced a seizure of that particular type. In particular embodiments, the neural network may output the onset of a seizure and/or the duration of the seizure. In particular embodiments, the neural network may output a risk assessment or severity associated with the seizure. For example, different kinds of seizures are associated with different levels of risk of harm to the user. For instance, seizures while the user is asleep are correlated with sudden-death syndrome, and this syndrome is highly correlated with heart rhythm and blood oxygen during a seizure episode. If a seizure is sufficiently severe or is accompanied by a high level of risk to the user, then the system may automatically contact emergency contacts (e.g., police, ambulance services, or contacts specified by a user, etc.). For example, a wearable device with cellular or WiFi calling capabilities may request help for the user from emergency contacts, or a connected device (e.g., a smartphone or a server device) may make that request.

The neural network may include several different classifiers, each associated with a different type of seizure. For example, the neural network may include distinct classifiers, each one focused on a specific type of generalized seizures (e.g., absence seizures, tonic-clonic seizures, etc.) or a specific type of focal seizures (e.g., simple focal seizures, complex focal seizures, etc.). Thus, the output layer of the neural network may be comprised of the output layer of these classifiers. In particular embodiments, the neural network may include a general classifier that receives as input the output of each specific classifier and/or the sensor signals to make an overall determination of the whether or not the user suffered a seizure. For example, if a classifier for a specific type of seizure outputs a very high likelihood that the user suffered that particular type of seizure, then the overall likelihood that the user suffered a seizure may also be high. On the other hand, if multiple classifiers output a relatively lower likelihood that a user suffered a seizure (e.g., a 50% or so likelihood), then the overall likelihood that the user suffered a seizure may still be quite high.

In particular embodiments, in addition to the sensor signals that pass the stage-2 quality checks, input to a neural network may include information related to the user's seizure history. For example, a user's previous clinical diagnoses of certain types of seizures, and/or medical history related to the user's predisposition to certain type of seizures, may be inputs to the neural network, and features may be extracted from this data and used by the neural network to predict the user's current seizure condition.

In particular embodiments, output from stage 2 may be provided to a health care professional, such as the user's doctor, along with the underlying sensor signals (e.g., blood-oxygen levels, cardiac conditions, etc.) corresponding to the output. The health-care professional may evaluate and/or annotate the stage 2 output, for example by confirming the output, by adjusting the output, or by contradicting the output. For example, a doctor may review the underlying data (e.g., some or all of the sensor signals corresponding to the seizure condition output by stage 2) and conclude that that a seizure occurred and that the most likely seizure indicated by the stage 2 output was in fact the seizure that the user experienced. As another example, a doctor may agree that a seizure occurred but may conclude that the type of seizure is indeterminable, or that a different type of seizure likely occurred than the one indicated as most likely by stage 2 output. As another example, a doctor may determine that a seizure did or did not occur, or confirm or adjust an associated level of risk. This health-care professional feedback may be used, along with the corresponding sensor signals, to re-train the neural network, making it more reliable and specific to the particular user. In particular embodiments, as explained below, a stage 3 process may be used to identify the most relevant data for a health professional to review.

Prior to deployment, the neural network is trained using a large dataset of training data that includes sensor signals from the various sensor modalities used by the wearable device and corresponding ground-truth labeled data (e.g., labeled data indicating a seizure, a type of seizure, a duration of a seizure, etc.). Sensor signals may be based on a reference device, which may use the same hardware implementation as used by the wearable device or may use a different hardware implementation (e.g., training data for SpO₂ signals may be based on a pulse oximeter rather than a PPG optical sensor, in particular embodiments); however, the same features are extracted from the training sensor signals as are extracted from the wearable device senor signals during inference. As described herein, after initial training is complete, then the neural network may be deployed, and the neural network may be subsequently trained or fine-tuned based on user-specific data or based on updated generalized data (e.g., annotated data from many health-care professional assessing many different patients).

In particular embodiments, output from the stage-2 neural network may be a passed to a stage-3 neural-network discriminator, such as discriminator 250 of the example of FIG. 2. In particular embodiments, the stage 3 discriminator may be deployed on a cloud device or on an edge device, e.g., on the wearable device or an associated client device. The trained discriminator receives as input the output from the stage 2 neural network, and the stage-3 discriminator then prioritizes the stage-2 information for review by a medical professional. For example, some epilepsy patients may have many seizures, to the extent that reviewing data from each seizure is not feasible. Therefore, the stage-3 discriminator may identify whether a particular seizure condition should be given review priority, e.g., is a relatively severe seizure or is evidence of a severe condition (e.g., seizure data that occurs at night, which can correlate with sudden death syndrome). In addition to stage-2 output, the stage-3 discriminator may also receive as input data from the user's health records to determine the priority of stage-2 data, and may also receive sensor signals themselves, in particular embodiments. In particular embodiments, a stage-3 discriminator may serve to validate or falsify output from the stage-2 neural network.

The stage-3 discriminator may be trained on generalized user data from many users. Over time, the stage 3 discriminator for a particular user may be fine-tuned based on the user's medical history and input from a health professional. For example, indications by a health professional confirming, adjusting, or contradicting the seizure data from stage 2 or the priority determinations from stage 3 may be used to fine-tune the stage 3 model for that particular user.

In particular embodiments, different stages may activate different sensors. For example, a PPG sensor tends to be more power hungry than the other sensors discussed herein. Therefore, in particular embodiments, a PPG sensor may only be activated for short durations during stage 1. However, if the wearable activates stage 2, indicating that a user is suffering a seizure, then the PPG senor may be activated to continuously record data.

As explained above, the multi-stage techniques described herein provide both high sensitivity and high specificity for seizure detection. In combination with a wearable device equipped with multiple sensors, the techniques described herein provide for passive, continuous monitoring for seizure onset, followed by a robust determination of seizure occurrence, type, and severity. Moreover, the techniques described herein provide for seizure monitoring at times when human monitoring is difficult, e.g., while a user is asleep, which corresponds with some of the most severe outcomes (e.g., sudden-death syndrome). In addition, the techniques described herein provide for detection of seizures that have few, if any, symptoms that are observable by a human.

As described above, in particular embodiments, the techniques described herein detect seizure onset, seizure duration, and likely seizure type, and can record multiple seizure-related physiological signals before, during, and after seizure occurs, e.g., oxygen level, heart rate/rhythm, shaking frequency, etc. This data can be provided to a user or to a health professional. For example, seizure frequency and duration are crucial for seizure management and seizure treatment, and blood oxygen and heart rhythm during a seizure episode are highly related to SUDEP, and therefore receiving information describing a user's seizure conditions and the corresponding physiological measurements can greatly improve the ability of a health professional to track, diagnose, and optimally treat a person's seizures and epilepsy. In addition, the medical causes of epilepsy and seizures are not well understood, and the techniques described herein can monitor and obtain seizure-related data while users go about their daily lives, without the requiring the user to visit a clinic to be monitored by medical professionals.

When a seizure is detected, an alert can be generated to the user, to the user's medical professionals, to an emergency contact, and/or to emergency services. Depending on the user preferences, seriousness of the seizure, and the urgency for intervention, an alert with geolocation can be immediately sent to a medical professional and/or to emergency services as a seizure is occurring. The seriousness and urgency related to a seizure can be defined by criteria including duration of the seizure, lack of user input after the seizure onset (which is an indication of unconsciousness), a dangerous level of blood oxygen level, a dangerous heart rhythm, and/or a fall detection. In particular embodiments, a user can enable the cloud-based discriminator so that the detailed measurement of the detected episode will be routed to the discriminator for legitimacy and urgency confirmation before sending out the alert, i.e., the stage-3 system may provide a check on the stage-2 results, reducing the incidence of false alerts.

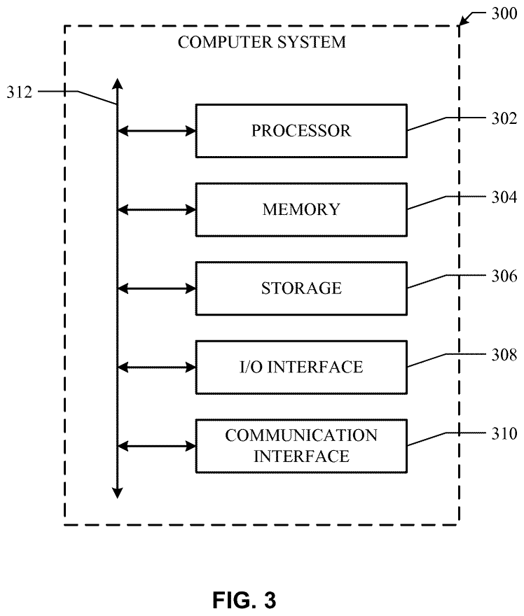

FIG. 3 illustrates an example computer system 300. In particular embodiments, one or more computer systems 300 perform one or more steps of one or more methods described or illustrated herein. In particular embodiments, one or more computer systems 300 provide functionality described or illustrated herein. In particular embodiments, software running on one or more computer systems 300 performs one or more steps of one or more methods described or illustrated herein or provides functionality described or illustrated herein. Particular embodiments include one or more portions of one or more computer systems 300. Herein, reference to a computer system may encompass a computing device, and vice versa, where appropriate. Moreover, reference to a computer system may encompass one or more computer systems, where appropriate.

This disclosure contemplates any suitable number of computer systems 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 300 may include one or more computer systems 300; be unitary or distributed; span multiple locations; span multiple machines; span multiple data centers; or reside in a cloud, which may include one or more cloud components in one or more networks. Where appropriate, one or more computer systems 300 may perform without substantial spatial or temporal limitation one or more steps of one or more methods described or illustrated herein. As an example and not by way of limitation, one or more computer systems 300 may perform in real time or in batch mode one or more steps of one or more methods described or illustrated herein. One or more computer systems 300 may perform at different times or at different locations one or more steps of one or more methods described or illustrated herein, where appropriate.

In particular embodiments, computer system 300 includes a processor 302, memory 304, storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 302 includes hardware for executing instructions, such as those making up a computer program. As an example and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306. In particular embodiments, processor 302 may include one or more internal caches for data, instructions, or addresses. This disclosure contemplates processor 302 including any suitable number of any suitable internal caches, where appropriate. As an example and not by way of limitation, processor 302 may include one or more instruction caches, one or more data caches, and one or more translation lookaside buffers (TLBs). Instructions in the instruction caches may be copies of instructions in memory 304 or storage 306, and the instruction caches may speed up retrieval of those instructions by processor 302. Data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches may speed up read or write operations by processor 302. The TLBs may speed up virtual-address translation for processor 302. In particular embodiments, processor 302 may include one or more internal registers for data, instructions, or addresses. This disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In particular embodiments, memory 304 includes main memory for storing instructions for processor 302 to execute or data for processor 302 to operate on. As an example and not by way of limitation, computer system 300 may load instructions from storage 306 or another source (such as, for example, another computer system 300) to memory 304. Processor 302 may then load the instructions from memory 304 to an internal register or internal cache. To execute the instructions, processor 302 may retrieve the instructions from the internal register or internal cache and decode them. During or after execution of the instructions, processor 302 may write one or more results (which may be intermediate or final results) to the internal register or internal cache. Processor 302 may then write one or more of those results to memory 304. In particular embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which may each include an address bus and a data bus) may couple processor 302 to memory 304. Bus 312 may include one or more memory buses, as described below. In particular embodiments, one or more memory management units (MMUs) reside between processor 302 and memory 304 and facilitate accesses to memory 304 requested by processor 302. In particular embodiments, memory 304 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 304 may include one or more memories 304, where appropriate. Although this disclosure describes and illustrates particular memory, this disclosure contemplates any suitable memory.

In particular embodiments, storage 306 includes mass storage for data or instructions. As an example and not by way of limitation, storage 306 may include a hard disk drive (HDD), a floppy disk drive, flash memory, an optical disc, a magneto-optical disc, magnetic tape, or a Universal Serial Bus (USB) drive or a combination of two or more of these. Storage 306 may include removable or non-removable (or fixed) media, where appropriate. Storage 306 may be internal or external to computer system 300, where appropriate. In particular embodiments, storage 306 is non-volatile, solid-state memory. In particular embodiments, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 may include one or more storage control units facilitating communication between processor 302 and storage 306, where appropriate. Where appropriate, storage 306 may include one or more storages 306. Although this disclosure describes and illustrates particular storage, this disclosure contemplates any suitable storage.

In particular embodiments, I/O interface 308 includes hardware, software, or both, providing one or more interfaces for communication between computer system 300 and one or more I/O devices. Computer system 300 may include one or more of these I/O devices, where appropriate. One or more of these I/O devices may enable communication between a person and computer system 300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 308 for them. Where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In particular embodiments, communication interface 310 includes hardware, software, or both providing one or more interfaces for communication (such as, for example, packet-based communication) between computer system 300 and one or more other computer systems 300 or one or more networks. As an example and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it. As an example and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 300 may include any suitable communication interface 310 for any of these networks, where appropriate. Communication interface 310 may include one or more communication interfaces 310, where appropriate. Although this disclosure describes and illustrates a particular communication interface, this disclosure contemplates any suitable communication interface.

In particular embodiments, bus 312 includes hardware, software, or both coupling components of computer system 300 to each other. As an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 312 may include one or more buses 312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
detecting, by each of a plurality of sensors of a wearable device worn by a user, a physiological signal of the user;
determining, by the wearable device, whether at least one of the detected physiological signals indicates that the user is suffering a seizure; and
in response to a determination that the user is suffering a seizure, then determining, by a trained neural network and based on a plurality of the detected physiological signals, a seizure condition of the user.

2. The method of claim 1, wherein the plurality of sensors comprises at least two of: an accelerometer, an EMG sensor, a skin-impedance sensor, an SpO₂ sensor, and a PPG sensor.

3. The method of claim 1, wherein determining whether at least one of the detected physiological signals indicates that the user is suffering a seizure comprises determining that at least one of the detected physiological signals meets a predetermined threshold specific to that physiological signal.

4. The method of claim 3, wherein the predetermined threshold is defined at least in part by one or more user characteristics of the user.

5. The method of claim 1, further comprising:
determining, by the wearable device, whether each of the detected physiological signals meets a corresponding first signal-quality threshold; and
in response to a determination that a detected physiological signal does not meet a corresponding first signal-quality threshold, then discarding that physiological signal prior to determining whether at least one of the detected physiological signals indicates that the user is suffering a seizure.

6. The method of claim 5, further comprising:
determining, by the wearable device, whether each of the detected physiological signals meets a corresponding second signal-quality threshold; and
in response to a determination that a detected physiological signal does not meet a corresponding second signal-quality threshold, then discarding that physiological signal prior to determining a seizure condition of the user.

7. The method of claim 1, wherein determining a seizure condition of the user comprises determining, by the neural network, whether the user suffered a seizure.

8. The method of claim 7, wherein determining a seizure condition of the user further comprises determining one or more of (1) an onset of the seizure and (2) a duration of the seizure.

9. The method of claim 1, wherein determining a seizure condition of the user comprises classifying, by the neural network, a type of seizure suffered by the user.

10. The method of claim 9, further comprising determining, for each of a plurality of types of seizures, a probability that the user suffered that particular type of seizure.

11. The method of claim 10, wherein the probability is determined at least in part on the user's seizure history.

12. The method of claim 1, wherein the trained neural network is deployed on the wearable device.

13. The method of claim 1, further comprising determining, by a trained discriminator and based on the determined seizure condition, a priority of the seizure condition.

14. The method of claim 13, wherein the priority of the seizure condition is further determined based on the user's seizure history.

15. The method of claim 14, wherein the user's seizure history comprises input from a health-care provider.

16. The method of claim 15, further comprising finetuning the trained neural network based at least in part on the input from the health-care provider.

17. The method of claim 1, further comprising providing, to the user, a notification regarding the seizure condition.

18. The method of claim 1, further comprising:
determining a severity of the seizure condition of the user; and
making, based on the determined severity, a request for emergency aid for the user.

19. A wearable device comprising:
a plurality of sensors, each sensor configured to detect a physiological signal of a user wearing the wearable device;
one or more non-transitory computer readable storage media storing instructions; and one or more processors coupled to the one or more non-transitory computer readable storage media and operable to execute the instructions to:
access, from each of the plurality of sensors, a respective detected physiological signal of the user;
determine whether at least one of the detected physiological signals indicates that the user is suffering a seizure; and
in response to a determination that the user is suffering a seizure, then determine, by a trained neural network and based on a plurality of the detected physiological signals, a seizure condition of the user.

20. An apparatus comprising:
a wearable device comprising:
a plurality of sensors, each sensor configured to detect a physiological signal of a user wearing the wearable device;
one or more first non-transitory computer readable storage media storing instructions; and one or more first processors coupled to the one or more first non-transitory computer readable storage media and operable to execute the instructions to:
access, from each of the plurality of sensors, a respective detected physiological signal of the user;
determine whether at least one of the detected physiological signals indicates that the user is suffering a seizure; and
in response to a determination that the user is suffering a seizure, then provide, to a computing device, one or more of the detected physiological signals; and
the computing device, comprising one or more second non-transitory computer readable storage media storing instructions; and one or more second processors coupled to the one or more second non-transitory computer readable storage media and operable to execute the instructions to:
access the one or more detected physiological signals provided by the wearable device; and
determine, by a trained neural network on the computing device and based on a plurality of the accessed physiological signals, a seizure condition of the user.
